(19) United States
(12) Patent Application Publication
(10) Pub. No.: US 2003/0045856 A1
(43) Pub. Date: Mar. 6, 2003

(54) Mechanical fastening system having orthogonally oriented engagement members

(76) Inventors: Denise R. Couture, Appleton, WI (US); Yvette L. Hammonds, Oshkosh, WI (US); Valerie L. Kurbec, Neenah, WI (US); Shelley R. Rasmussen, Oshkosh, WI (US); William G. Reeves, Appleton, WI (US); William G. Stratton, Anoka, MN (US); Manuel A. Torres, Cumming, GA (US)

Correspondence Address: KIMBERLY-CLARK WORLDWIDE, INC., 401 NORTH LAKE STREET, NEENAH, WI, 54956

(21) Appl. No.: 10/037,287

(22) Filed: Dec. 20, 2001

Related U.S. Application Data
(60) Provisional application No. 60/313,604, filed on Aug. 20, 2001.

(51) Int. Cl. A61F 13/15; A61F 13/20; A44B 1/04; A44B 11/25; A44B 17/00; A44B 18/00

(52) U.S. Cl. 604/391; 604/385.04; 604/385.13; 604/387; 24/442

(57) ABSTRACT

A selectively releasable, interengaging fastening system that may be used for a variety of articles such as, for example, shoes, garments, disposable absorbent products such as disposable diapers, sanitary napkins, incontinence products and the like. The fastening system includes a first fastener component that can constitute at least a first portion of an article. The first fastener component includes an engagement section having a plurality of substantially non-isotropic engagement members such that the engagement section has an axis of substantially maximal engagement. The fastening system also includes a cooperating fastener component that may constitute at least a second portion of an article such that the first and second portions of the article are capable of being joined by movement of the respective components together generally along an attachment direction into an overlapping and interengaging configuration. The first fastener component is oriented so its axis of substantially maximal engagement is generally orthogonal to the attachment direction. This configuration causes the first fastener component to become more interengaged with the cooperating fastener component as the article is subjected to various forces such as, for example, the type of forces encountered by the fastening system when used on a disposable personal care product that is worn.

### FIELD OF THE INVENTION

The present invention relates to fastening systems for garments and other articles. More particularly, the present invention relates to interlocking, mechanical-type fastening systems which can be employed with disposable articles, such as gowns, diapers, incontinence garments and the like.

### BACKGROUND

Absorbent personal care articles such as diapers, sanitary napkins, incontinence pads and the like may be secured around a wearer or to an undergarment to hold the article in proper position during use. These articles frequently employ adhesive tabs, wings or flaps, garment attachment adhesives and/or similar systems. If the article is secured to an undergarment, it may employ wings or flaps that use adhesive to secure the end of the wing or flap to the undergarment.

Even with the combination of wings/flaps and garment attachment adhesive, users may encounter difficulty keeping the article in position. This problem is particularly apparent when the user is physically active or when heat, moisture and/or humidity weaken the adhesive used to secure the article.

While different types of attachment systems such as belts, snaps or the like are known, these systems increase the cost and complexity of the article as well as difficulty for the user to attach or apply the absorbent article.

Mechanical fastening systems appear on some commercially available personal care articles. Typically, a conventional mechanical fastening system of the hook and loop type is used on flaps or wings of a product. However, these conventional systems may become disengaged when stresses are applied to the article over time. These stresses can be caused by a wide range of movements by the wearer, unusual movements, intense activity such as exercise, and/or bunching and twisting of the personal care product.

### SUMMARY

The present invention addresses the problems discussed above by providing a selectively releasable, interengaging fastening system that may be used for a variety of articles such as, for example, shoes, garments, disposable absorbent products such as disposable diapers, sanitary napkins, incontinence products and the like.

The fastening system includes a first fastener component that can constitute at least a first portion of an article. The first fastener component includes an engagement section having a plurality of substantially non-isotropic engagement members such that the engagement section has an axis of substantially maximal engagement.

The fastening system also includes a cooperating fastener component that may constitute at least a second portion of an article such that the first and second portions of the article are capable of being joined by movement of the respective components together generally along an attachment direction into an overlapping and interengaging configuration.

According to the present invention, the first fastener component is oriented so its axis of substantially maximal engagement is generally orthogonal to the attachment direction. While the inventors should not be held to any particular theory of operating, it is believe that this configuration causes the first fastener component to become more interengaged with the cooperating fastener component as the article is subjected to various forces such as, for example, the type of forces encountered by the fastening system when used on a disposable personal care product that is worn.

In an aspect of the invention, each substantially non-isotropic engagement member may have a stem portion with a distal end region and a securement element disposed at the distal end region of its corresponding stem portion.

Desirably, the engagement section has an axis of maximal engagement and a generally perpendicular axis of minimal engagement.

In one embodiment of the invention, the cooperating fastener component may be substantially non-isotropic and includes an axis of substantially maximal engagement. For example, the axis of substantially maximal engagement of the substantially non-isotropic engagement section and the axis of substantially maximal engagement of the substantially non-isotropic cooperating fastener component may both be substantially parallel and each may be oriented generally orthogonal to the attachment direction.

According to the invention, at least one of the first portion and the second portion of the article on which the fastening system is placed or included may be a segment having a fixed end attached to an article body and a free end.

Generally speaking, the cooperating fastener component may be a loop material such as, for example, a woven, knit or a nonwoven loop material. For example, the nonwoven loop material may be a pattern unbonded material such as, for example, the material described in U.S. Pat. Nos. 5,858,515 and/or 5,763,041, the contents of which are incorporated herein by reference.

In an aspect of the invention, fastening system can also be used to secure an article in a configuration convenient for disposal.

In another aspect of the invention, the fastening system also provides ease of use or application benefits. In particular, the wings may be engaged on the topsheet side or body side of the sanitary napkin or incontinence article while the peel strip protecting the garment adhesive is removed. The article may then me placed in position on an undergarment and then the wings may be engaged.

The present invention also encompasses an article that includes the fastening system. The article may have a lengthwise longitudinal direction, a lateral cross-direction, and a longitudinally extending medial line. The article may include a first article portion; a second article portion; and at least one selectively releasable, interengaging fastener system for securing the first article portion to the second article portion as described herein.

The selectively releasable, interengaging fastener system may include: (a) at least one first fastener component that may form at least one section of the first article portion, the first fastener component including an engagement section having a plurality of substantially non-isotropic engagement members such that the engagement section has an axis of substantially maximal engagement; and (b) at least one cooperating fastener component that may form at least one section of the second article portion such that the first and second article portions are capable of being joined by movement of the respective components together generally along an attachment direction into an overlapping and interengaging configuration. According to the present invention, the first fastener component is oriented so its axis of substantially maximal engagement is generally orthogonal to the attachment direction.

Desirably, the first fastener component may extend across the width and/or length dimension of the first article portion. For example, if the first article portion is a wing or flap, the first fastener component may extend across the wing or flap generally along the longitudinal axis of the product. More desirably, the first fastener component may be a flexible component that extends across the wing or flap and is flexible enough to fold, bend or otherwise flex with the flap or wing.

The article may further include substantially non-isotropic engagement members having a stem portion with a distal end region and a securement element disposed at the distal end region of its corresponding stem portion. The engagement section of the first fastener component may have an axis of maximal engagement and a generally perpendicular axis of minimal engagement. The cooperating fastener component may also be substantially non-isotropic and have an axis of substantially maximal engagement.

At least one of the first article portion and the second article portion may be a tab, ear, flap or wing-like element or segment having a free end and a fixed end attached to an article body.

In one embodiment, the first article portion may provide a first waistband portion. The second article portion may provide a second waistband portion. The article may also have an intermediate portion which interconnects said first and second waistband portions. The article may further include a backsheet layer; a substantially liquid-permeable topsheet layer; and an absorbent body sandwiched between said backsheet layer and topsheet layer. Generally speaking, this article may be in the form of a disposable diaper, incontinence pant, training pant or similar structure.

In yet another embodiment, the first article portion and the second article portion each may provide respective first and second flap portions, each flap portion having a free end and a fixed end attached to an intermediate portion which interconnects the first and second flap portions. The article may further include a backsheet layer; a substantially liquid-permeable topsheet layer; and an absorbent body sandwiched between said backsheet layer and topsheet layer. Generally speaking, this article may be in the form of a sanitary napkin, incontinence pad or similar structure.

Of course, the fastening system may also be adapted to secure the article in a configuration convenient for disposal.

The present invention also encompasses a selectively releasable, interengaging fastening system for a disposable sanitary napkin, The fastening system includes a first fastener component that may form at least a first portion of the sanitary napkin. The first fastener component may include an engagement section having a plurality of substantially non-isotropic engagement members such that the engagement section has an axis of substantially maximal engagement. The fastening system may also include a cooperating fastener component that may form at least a second portion of the sanitary napkin such that the first and second portions of the sanitary napkin are capable of being joined by movement of the respective components together generally along an attachment direction into an overlapping and interengaging configuration. According to the invention, the first fastener component is oriented so its axis of substantially maximal engagement is generally orthogonal to the attachment direction.

In an embodiment of the invention, the first portion and the second portion of the sanitary napkin may each provide respective first and second flap portions of the sanitary napkin. Each flap portion may have a free end and a fixed end attached to an intermediate portion that interconnects the first and second flap portions. The sanitary napkin may further include a backsheet layer; a substantially liquid-permeable topsheet layer; and an absorbent body sandwiched between said backsheet layer and topsheet layer.

The present invention also encompasses a sanitary napkin that includes the selectively releasable, interengaging fastening system described herein.

In an embodiment, the selectively releasable, interengaging fastening system may include a first wing extending from the first longitudinal edge of the sanitary napkin and a second wing extending from the second longitudinal edge of the sanitary napkin. Each wing may have a fixed end and a free end.

A first fastener component may form at least a portion of at least the first wing. The first fastener component may an engagement section having a plurality of substantially non-isotropic engagement members such that the engagement section has an axis of substantially maximal engagement.

A cooperating fastener component may form at least a portion of at least the second wing such that the first and second wings of the sanitary napkin are capable of being joined by movement of the respective components together generally along an attachment direction into an overlapping and interengaging configuration. According to the invention, the first fastener component is oriented so its axis of substantially maximal engagement is generally orthogonal to the attachment direction.

In an aspect of the invention, both the first wing and the second wing may include the first fastener component and the second fastener component. In another aspect of the invention, at least one wing includes perforation lines adjacent its fixed end to provide quick removal of the napkin by tearing the wing along the perforation lines.

The present invention is also directed to a selectively releasable, interengaging fastening system for a disposable personal care product, in which the fastening system includes: a first fastener component comprising an engagement section having a plurality of engagement members; and a cooperating fastener component; such that the fastening system is adapted to become more interengaged as the disposable personal care product is worn.

### BRIEF DESCRIPTION OF THE DRAWINGS

The present invention will be more fully understood and further advantages will become apparent when reference is made to the following detailed description of the invention and the drawings, in which:

FIG. 1A is an illustration of an exemplary article incorporating an exemplary fastening system of the invention;

FIG. 1B is an illustration of an exemplary article incorporating an exemplary fastening system of the invention;

FIG. 1C is an illustration of an exemplary article incorporating an exemplary fastening system of the invention as it is used with an undergarment;

FIG. 1D is an illustration of an exemplary article incorporating an exemplary fastening system of the invention—with the backsheet of the article facing upward;

FIG. 1D′ is an illustration of an exemplary article incorporating an exemplary fastening system of the invention—with the backsheet of the article facing upward;

FIG. 1E is an illustration of a portion of an exemplary article incorporating an exemplary fastening system of the invention—with the backsheet of the article facing upward;

FIG. 2A is an illustration of an exemplary article incorporating an exemplary fastening system of the invention.

FIG. 2B is an illustration of an exemplary article incorporating an exemplary fastening system of the invention as it would be fastened to form a diaper, training pant or large incontinence article.

FIG. 3A is an illustration of an exemplary, non-isotropic engagement member which can be employed with the present invention;

FIG. 3B is an illustration of an exemplary, non-isotropic engagement member which can be employed with the present invention;

FIG. 3B′ representatively shows a top view of the engagement member of FIG. 3B;

FIG. 3C is an illustration of an exemplary, non-isotropic engagement member which can be employed with the present invention;

FIG. 3C′ representatively shows a top view of the engagement member of FIG. 3C;

FIG. 3D is an illustration of an exemplary, non-isotropic engagement member which can be employed with the present invention;

FIG. 3E is an illustration of an exemplary, non-isotropic engagement member which can be employed with the present invention;

FIGS. 4A-B are graphical representations of data in Table 1;

FIGS. 5A-B are graphical representations of data in Table 1;

FIGS. 6A-B are photomicrographs of an exemplary, non-isotropic engagement member which can be employed with the present invention;

FIGS. 7 and 8 are photomicrographs of exemplary cooperating fastener members (e.g., loop materials) that may be used with the present invention;

### DETAILED DESCRIPTION OF THE INVENTION

The various aspects and embodiments of the invention will be described in the context of a disposable absorbent article, such as a sanitary napkin or a disposable diaper. It is, however, readily apparent that the present invention could also be employed with other articles, such as caps, gowns, shoe covers, feminine care articles, children's training pants, incontinence garments and the like. Typically, the disposable articles are intended for limited use and are not intended to be laundered or otherwise cleaned for reuse. A disposable diaper, for example, is discarded after it has become soiled by the wearer.

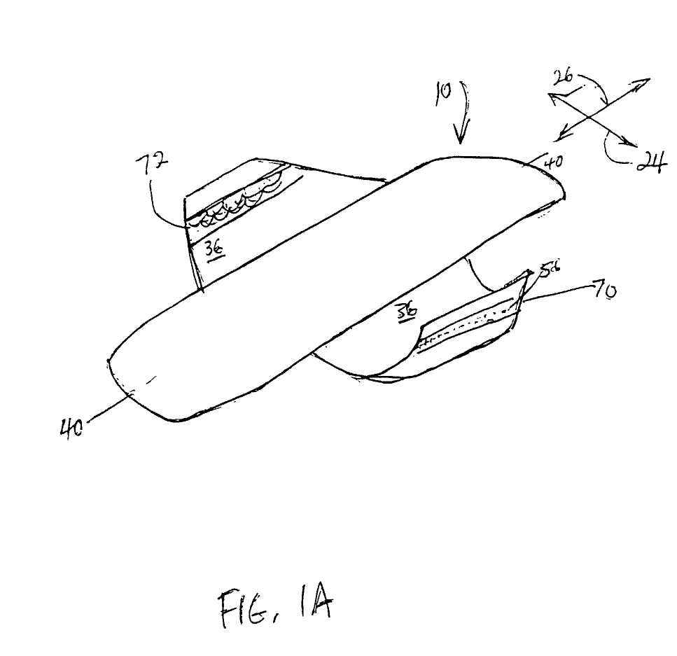

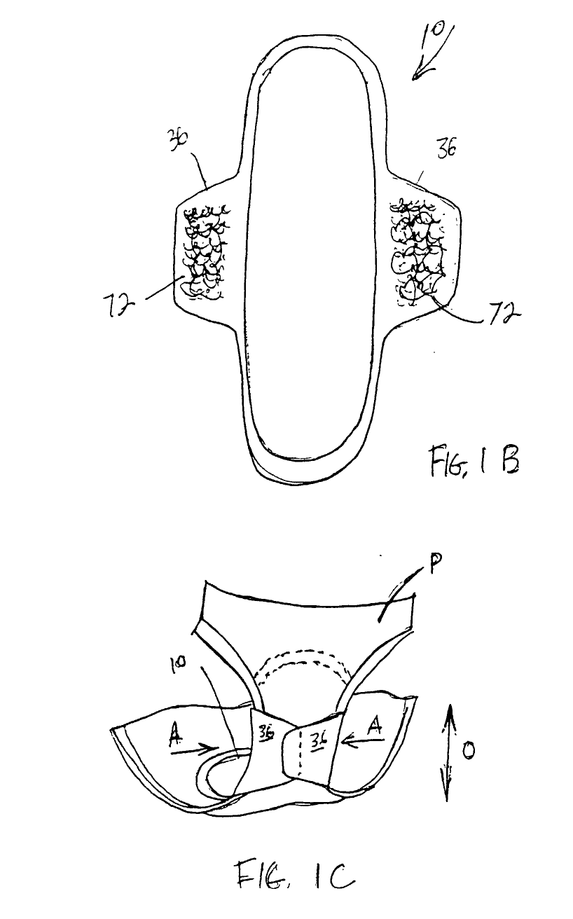

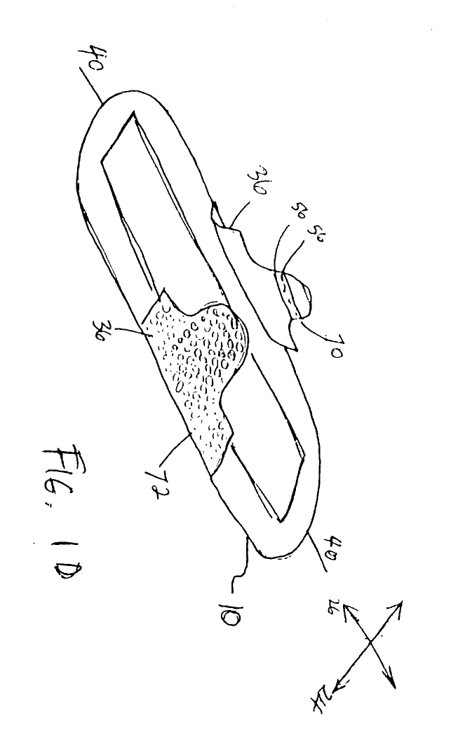

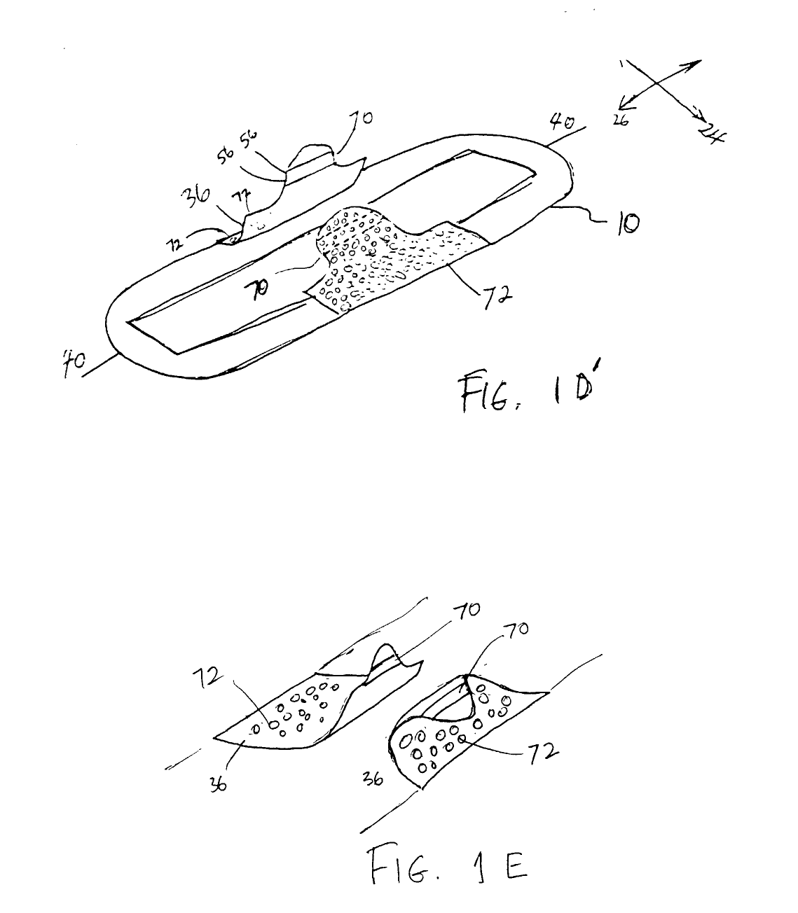

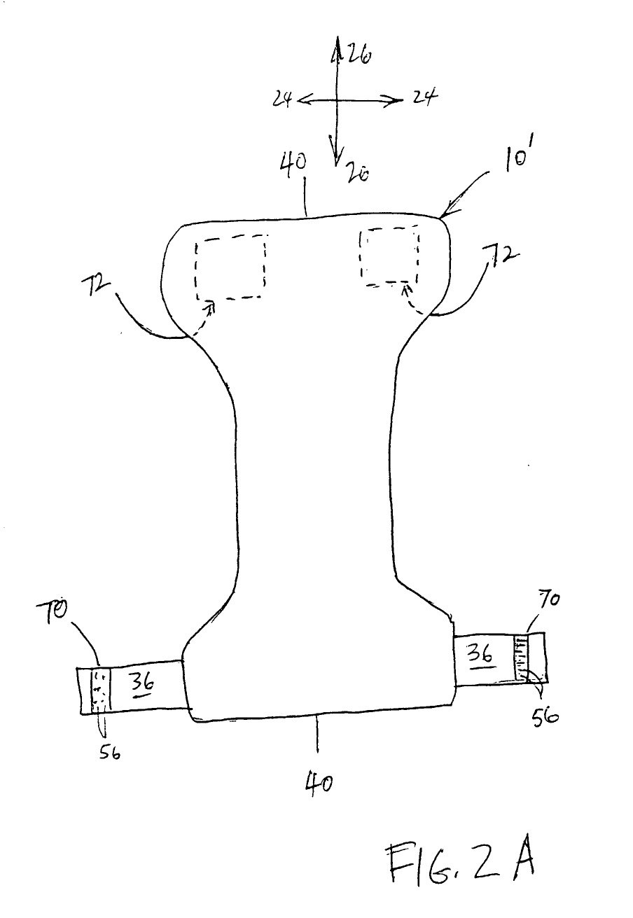

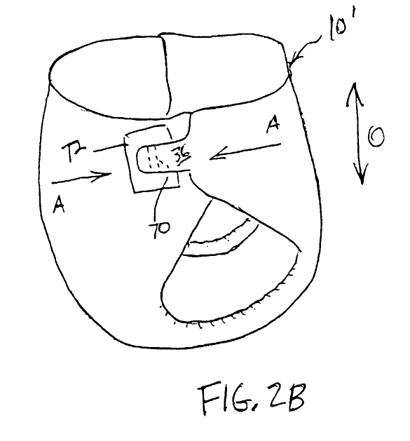

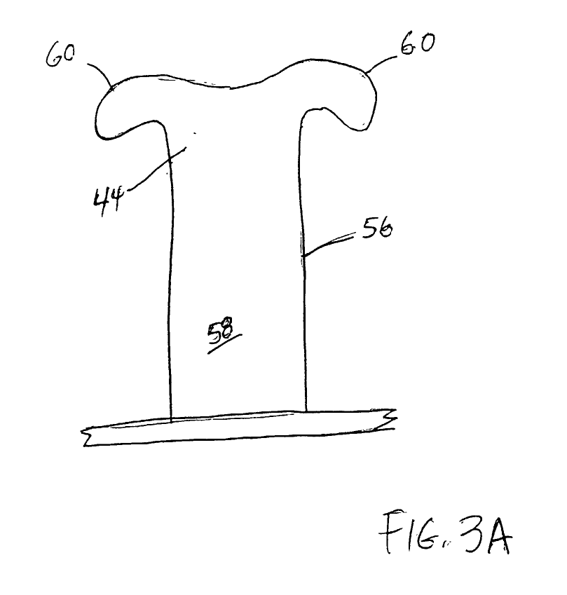

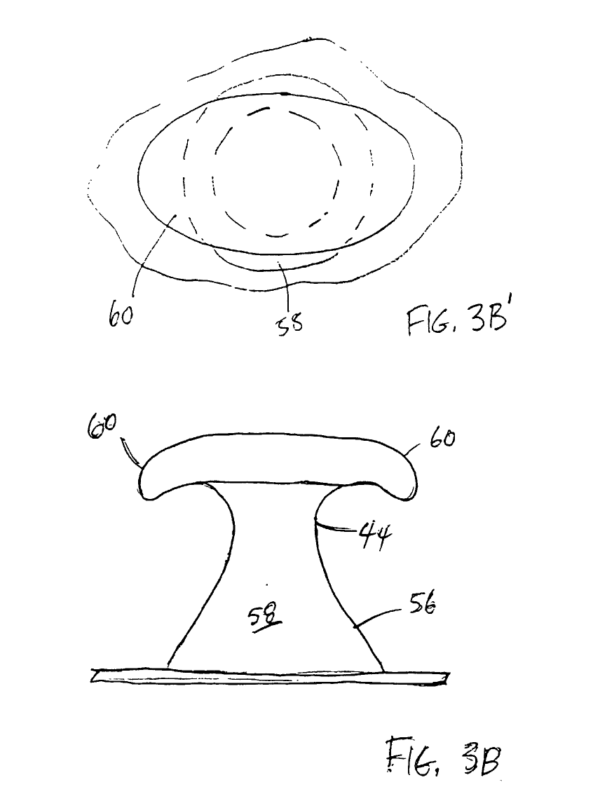

With reference to the Figures, an article, such as a sanitary napkin 10 illustrated in FIGS. 1A-E (or a disposable diaper 10′ illustrated in FIGS. 2A-B) has a lengthwise, longitudinal direction 26, a lateral cross-direction 24, and a longitudinally extending medial line 40. The article includes a first article portion, a second article portion and at least one fastener 36 for securing the first article portion to the second article portion. Such securement can, for example, be configured to thereby hold the article on a wearer. The fastener desirably includes at least one, first fastener component 70 attached to an appointed section of the first (or second) article portion, and a cooperating, second fastener component 72 attached to the second (or first) article portion. The first fastener component 70 includes an engagement section having a first plurality of engagement members 56. Each engagement member 56 has a stem portion 58 with a distal end region 44, and has at least one securement element 60 disposed at its corresponding distal end region. The plurality of engagement members has an arrangement pattern of their securement elements. It is contemplated that multiple pluralities of engagement members, each with different arrangement pattern of their securement elements, may be used.

Another aspect of the invention can provide an article in which the fastener component may include an engagement section having a plurality of non-isotropic engagement members. Each non-isotropic engagement member can have a stem portion 58 with a distal end portion 44, and a direction-dependent securement element 60 which is non-isotropically disposed at the distal end region of its corresponding stem portion 58 to provide a non-isotropic engagement opening. The plurality of non-isotropic engagement members can have an alignment pattern of their engagement openings. It is contemplated that multiple pluralities of non-isotropic engagement members may be used and that different alignment patterns of their engagement openings are possible.

In particular configurations, a majority of the plurality of non-isotropic engagement members have their engagement openings directed substantially orthogonal to an attachment direction. Generally speaking, the attachment direction is the direction in which the respective first and second portions of the article are brought together into an overlapping and interengaging relationship. Thus, in FIGS. 1 and 2, the attachment direction is generally a direction having a cross-directional vector-component along the lateral direction 24 and toward the medial line 40 of the article. Accordingly, the plurality of non-isotropic engagement members would have their engagement openings directed substantially parallel with the medial line 40 of the article.

In the various aspects of the invention, the individual engagement members are typically flexible and resilient, but will substantially retain their initial shape during ordinary use. When flexed or deformed during ordinary use, the engagement members will substantially avoid plastically deforming to sustain the deformation, and will, instead, substantially return or “spring-back” to their original orientations and shape.

The various aspects (individually and in combination) of the present invention can advantageously help to better maintain the desired fit around the wearer. For example, the aspects of the invention can help reduce the sagging and drooping of the crotch region of the garment, and can help reduce roll-over and drooping at the waist region. The incorporation of the various aspects of the fastening system of the invention can provide improved securement with greater resistance to premature pop-opens, and can also help provide improved fit, greater comfort and reduced irritation of the wearer's skin. The article of the invention can, for example, be a such as a disposable diaper. In desired aspects of the invention, the first article portion can provide a first, back waistband portion, and the second article portion can provide a second, front waistband portion. In addition, the article can have an intermediate or crotch portion which interconnects between the first and second waistband portions, respectively. The diaper can further include a backsheet layer, a liquid permeable topsheet layer connected and assembled in facing relation with the backsheet layer, and an absorbent structure, such as a structure which includes absorbent body. The absorbent structure is sandwiched between the backsheet and topsheet layers, and is operably held therebetween. A fastening system, such as the system including fastener, is typically constructed and arranged to interconnect the first waistband portion with the second waistband portion to hold the article on a wearer. The fastening system can be operatively configured to join the first, back waistband portion in an overlapping relation with the second, front waistband portion in a back-to-front arrangement to thereby encircle the wearer's body and hold the diaper secure on the wearer during use. Optionally, the fastening system can employ fasteners that are configured to join the front waistband portion in an overlapping relation with the back waistband portion in a front-to-back arrangement to secure the diaper. In such optional arrangements, the front waistband region may be identified as the first waistband portion and the rear waistband region may be identified as the second waistband portion.

FIG. 1 is an illustration of an exemplary sanitary napkin with fasteners 36 in the form of wings or flaps. At least one first fastener component 70 is attached to the wing 36 and at least one cooperating fastener component 72 is attached to the opposite wing 36. In some embodiments of the invention, the first fastener component 70 and the cooperating fastener component 72 may be attached to each wing such that the wings may be fastened without concern for overlapping the wings in any particular order. In other yet embodiments, the wing may be formed partially or entirely of the cooperating fastener component 72. FIG. 1A is an illustration of the sanitary napkin with its wings 36 or flaps secured around an undergarment or panty “P”. The arrows labeled “A” generally represent the attachment direction. The arrows labeled “O” generally represent the direction that is orthogonal to the attachment direction. It should be understood that this orthogonal direction is thought to be generally or substantially along or in the plane of the article although in some specific cases, it include a minor Z-direction component.

According to the invention, the first fastener component should be configured to have an axis of maximal engagement. This can be accomplished by utilizing a plurality of engagement members 56 that are non-isotropic (i.e., anisotropic) or non-symmetric. When the axis of maximal engagement of the first fastener component is oriented to be generally orthogonal to the attachment direction (that is, the direction each fastener component is generally brought together to effect overlapping engagement), it has been unexpectedly found that the fastening system is adapted to become more interengaged as the product is worn. For example, peel force and shear force as determined utilizing standard test procedures prior to wearing the article is greater for fastening systems in which the axis of maximal engagement of the first fastener component is oriented in the attachment direction and lower for fastening systems in which the axis of maximal engagement of the first fastener component is oriented orthogonal to the attachment direction.

When the peel force and the shear force were measured after use, the values increased for both orientations. However, the peel force and sheer force values measured for fastening systems in which the axis of maximal engagement of the first fastener component is oriented orthogonal to the attachment direction were greater than those measured for fastening systems in which the axis of maximal engagement of the first fastener component is oriented in the attachment direction.

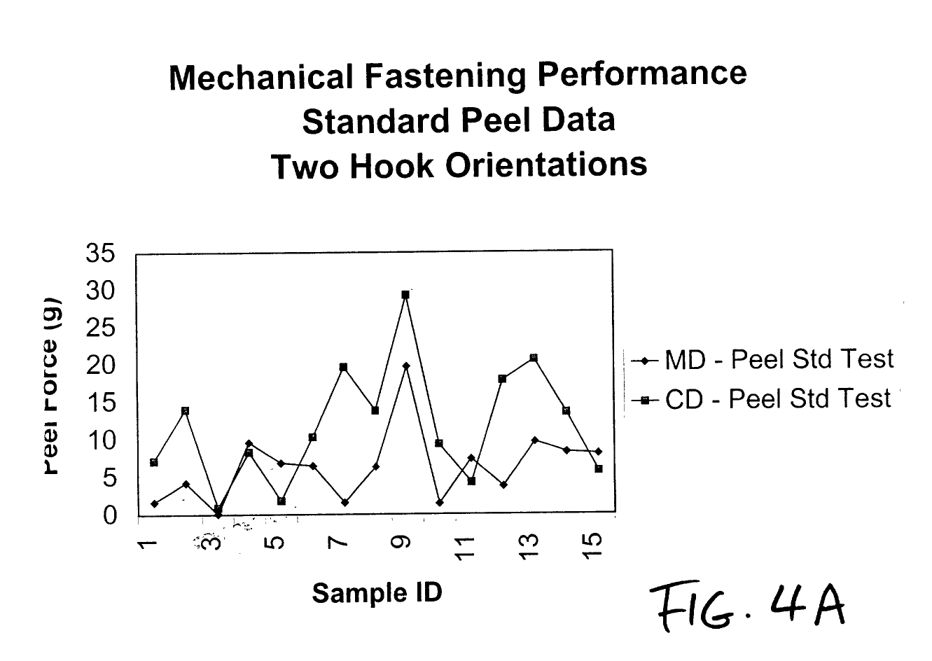

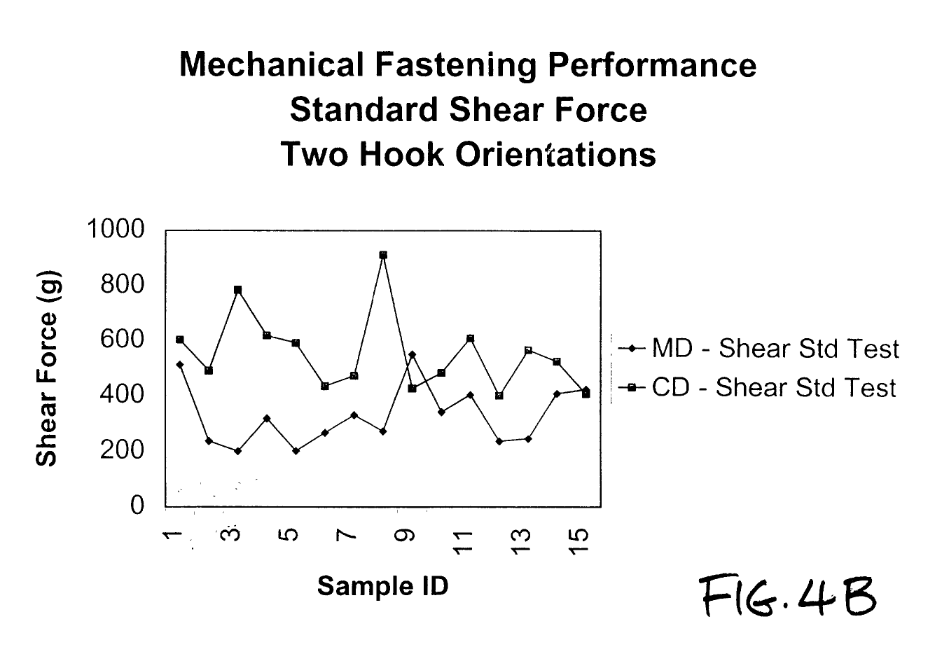

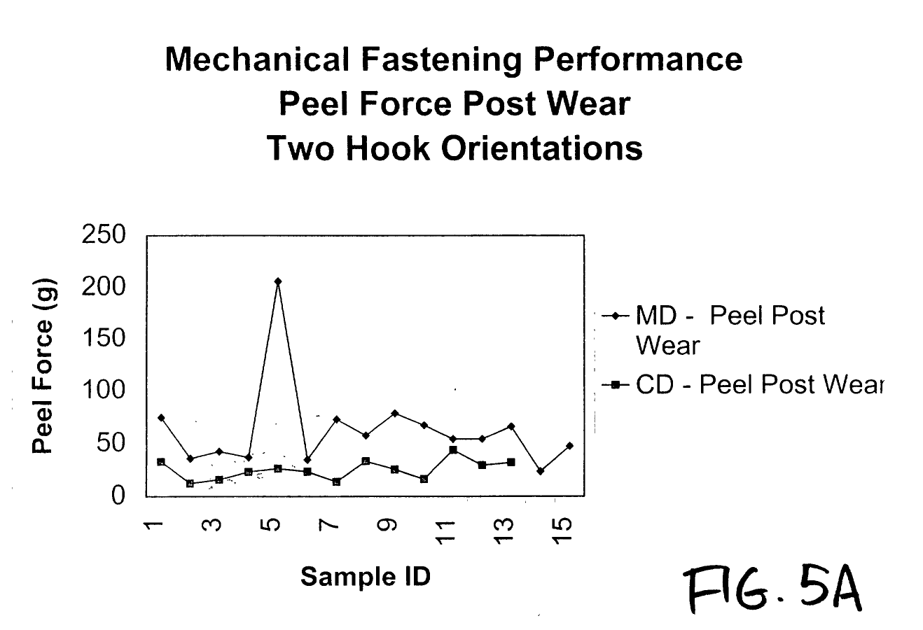

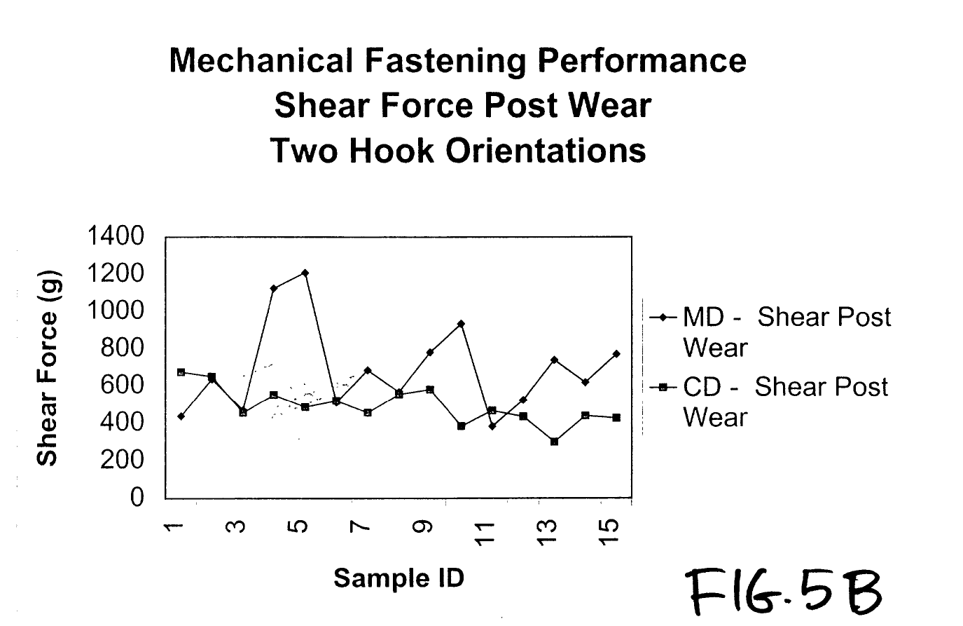

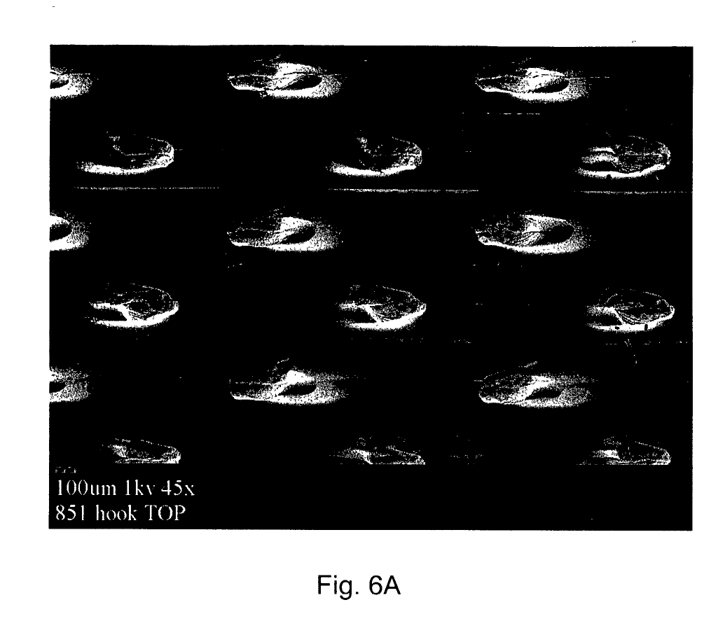

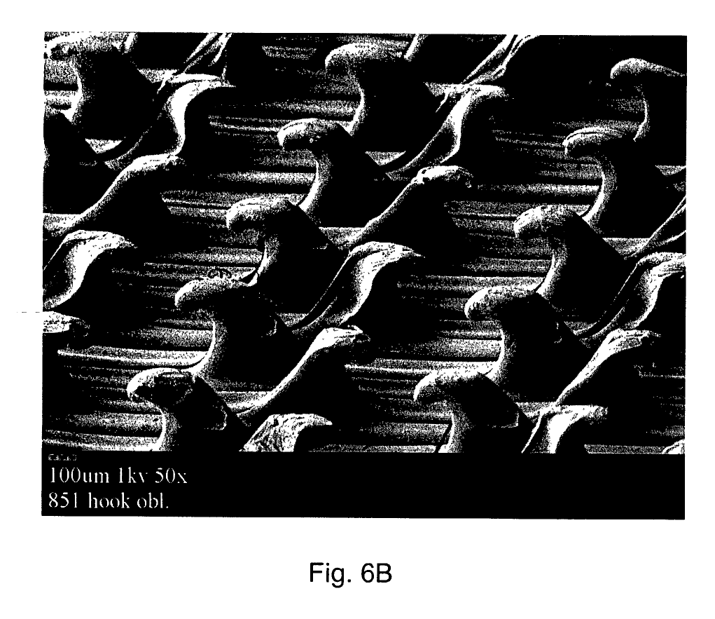

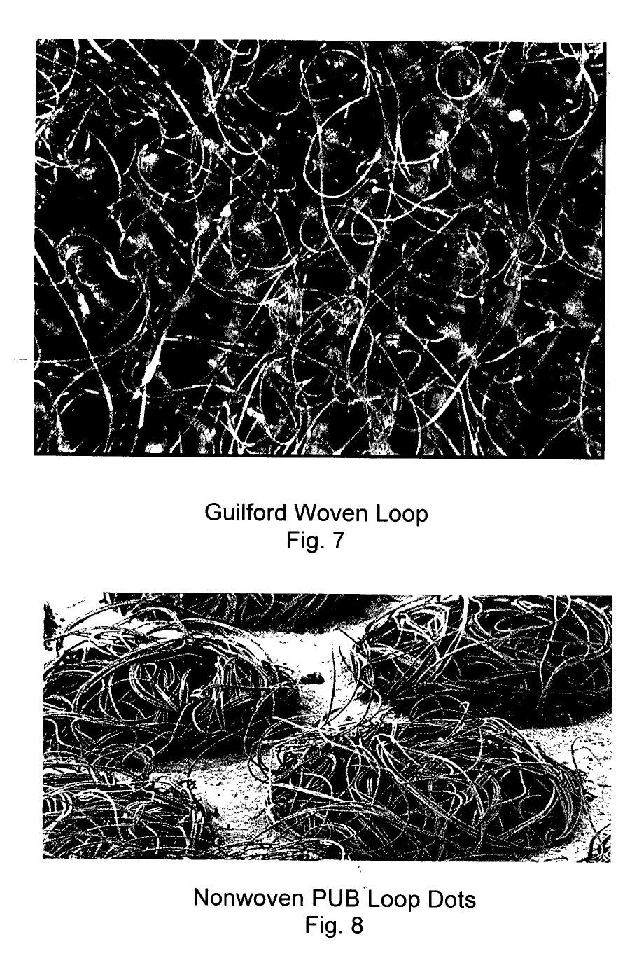

Data showing this result is in attached Table 1 and is shown graphically in FIGS. 4A-B and 5A-B.

The following is a brief description of the orientation direction with respect to the lengthwise, longitudinal direction 26 and the lateral cross-wise direction depicted in FIG. 1. In one exemplary sanitary napkin, the orienting the axis of maximal engagement of the first fastener component in the attachment direction meant orienting the first fastener component so its axis of maximal engagement was in the cross-machine direction or the lateral cross-direction 24 shown in FIG. 1. Thus, for that sanitary napkin, the orienting the axis of maximal engagement of the first fastener component generally orthogonal to the attachment direction meant orienting the first fastener component so its axis of maximal engagement was in the machine direction or the lengthwise, longitudinal direction 26 shown in FIG. 1.

FIG. 1D shows an embodiment of the invention in which a first fastener component 70 is affixed to a wing or flap 36 and a second fastener component 72 is affixed or integral with a corresponding wing or flap 36. FIG. 1D′ illustrates an embodiment in which a first fastener component is affixed to each wing or flap 36 and a second fastener component is present on each wing or flap 36 as well. FIG. 1E illustrates the embodiment in FIG. 1D′ such that the first fastener components 70 and the second fastener components 72 on each flap or wing 36 are more visible. More particularly, FIG. 1E shows a pair of wings or flaps 36 each having a first fastener component 70 affixed to the wing so as to face the baffle or peel strip of an article as well as a second fastener component 72 affixed to or integral with wing so as to face the opposite side of the wing (i.e., the side of the wind facing away from the baffle or peel strip).

FIG. 2 is an illustration of an exemplary diaper with fasteners 36 in the form of tabs or ears extending from a waistband section of the diaper. At least one first fastener component is attached to the ear 36 and at least one cooperating fastener component 72 is attached to a corresponding portion of the diaper so that the diaper can be configured for wearing as shown, for example, in FIG. 2A. The arrows labeled “A” generally represent the attachment direction.

The diaper 10 can typically include a porous, liquid permeable topsheet 28; a substantially liquid impermeable backsheet 30; an absorbent body structure 32 positioned and connected between the topsheet and backsheet; a surge management portion 46 located adjacent the absorbent structure; and a system of elastomeric gathering members, such as a system including leg elastics 34 and waist elastics 42. The surge management portion is positioned in a liquid communication with an appointed storage or retention portion of the absorbent structure, and the topsheet 28, backsheet 30, absorbent structure 32, surge management portion 46 and elastic members 34 and 42 may be assembled together into a variety of well-known diaper configurations. The diaper can additionally include a system of containment flaps 62, and a system of side panel or ear region members 38, which may be elasticized or otherwise rendered elastomeric.

Various techniques for forming the desired fastening systems are described in U.S. Pat. No. 5,399,219 of T. Roessler et al., entitled METHOD FOR MAKING A FASTENING SYSTEM FOR A DYNAMIC FITTING DIAPER which issued Mar. 21, 1995 (attorney docket No. 11,186); in U.S. patent application Ser. No. 286,086 of D. Fries, entitled A PROCESS FOR ASSEMBLING ELASTICIZED EAR PORTIONS and filed Aug. 3, 1994 (attorney docket No. 11,169) which corresponds to U.S. Pat. No. 5,540,796; and in U.S. patent application Ser. No. 08/415,383 of D. Fries, entitled AN ASSEMBLY PROCESS FOR A LAMINATED TAPE and filed Apr. 3, 1995 (attorney docket No. 11,950) which corresponds to U.S. Pat. No. 5,595,618. The disclosures of the above-described documents are incorporated herein by reference in a manner that is consistent (not in conflict) herewith.

The diaper 10 generally defines the longitudinally extending length direction 26 and the laterally extending width direction 24, as representatively shown in FIGS. 1 and 2. The diaper may have any desired shape, such as rectangular, I-shaped, a generally hourglass shape, or a T-shape. With the T-shape, the crossbar of the “T” may comprise the front waistband portion of the diaper, or may alternatively comprise the rear waistband portion of the diaper.

The topsheet 28 and backsheet 30 may be generally coextensive, and may have length and width dimensions which are generally larger than and extend beyond the corresponding dimensions of the absorbent structure 32 to provide for the corresponding side margins 20 and end margins 22. Optionally, the topsheet and backsheet layers may not be coextensive. The topsheet 28 is operatively associated with and superimposed on backsheet 30, thereby defining the periphery of the diaper. The waistband regions comprise those portions of the diaper, which when worn, wholly or partially cover or encircle the waist or mid-lower torso of the wearer. The intermediate, crotch region 16 lies between and interconnects the waistband regions 14 and 12, and comprises that portion of the diaper which, when worn, is positioned between the legs of the wearer and covers the lower torso of the wearer. Thus, the intermediate crotch region 16 is an area where repeated fluid surges typically occur in the diaper or other disposable absorbent article.

The backsheet 30 can typically be located along an outer-side surface of the absorbent body 32 and may be composed of a liquid permeable material, but desirably comprises a material which is configured to be substantially impermeable to liquids. For example, a typical backsheet can be manufactured from a thin plastic film, or other flexible, substantially liquid-impermeable material. As used in the present specification, the term “flexible” refers to materials which are compliant and which will readily conform to the general shape and contours of the wearer's body. Backsheet 30 prevents the exudates contained in absorbent body 32 from wetting articles, such as bedsheets and overgarments, which contact diaper 10. In particular embodiments of the invention, backsheet 30 can include a film, such as a polyethylene film, having a thickness of from about 0.012 millimeters (0.5 mil) to about 0.051 millimeters (2.0 mils). For example, the backsheet film can have a thickness of about 1.25 mil.

Alternative constructions of the backsheet may comprise a woven or non-woven fibrous web layer which has been totally or partially constructed or treated to impart the desired levels of liquid impermeability to selected regions that are adjacent or proximate the absorbent body. For example, the backsheet may include a gas-permeable, nonwoven fabric layer laminated to a polymer film layer which may or may not be gas-permeable. Other examples of fibrous, cloth-like backsheet materials can comprise a stretch thinned or stretch thermal laminate material composed of a 0.6 mil (0.015 mm) thick polypropylene blown film and a 0.7 ounce per square yard (23.8 gsm) polypropylene spunbond material (2 denier fibers). A material of this type forms the outercover of a HUGGIES SUPREME disposable diaper, which is commercially available from Kimberly-Clark Corporation. The backsheet 30 typically provides the outer cover of the article. Optionally, however, the article may include a separate outer cover component member which is additional to the backsheet.

The backsheet 30 may alternatively include a micro-porous, “breathable” material which permits gases, such as water vapor, to escape from the absorbent body 32 while substantially preventing liquid exudates from passing through the backsheet. For example, the breathable backsheet may be composed of a microporous polymer film or a nonwoven fabric which has been coated or otherwise modified to impart a desired level of liquid impermeability. For example, a suitable microporous film can be a PMP-1 material, which is available from Mitsui Toatsu Chemicals, Inc., a company having offices in Tokyo, Japan; or an XKO-8044 polyolefin film available from 3M Company of Minneapolis, Minn. The backsheet may also be embossed or otherwise provided with a pattern or matte finish to exhibit a more aesthetically pleasing appearance.

In the various configurations of the invention, where a component such as the backsheet 30 or the containment flaps 62 are configured to be permeable to gas while having a resistance and limited permeability to aqueous liquid, the liquid resistant material can have a construction which is capable of supporting a hydrohead of at least about 45 cm of water substantially without leakage therethrough. A suitable technique for determining the resistance of a material to liquid penetration is Federal Test Method Standard FTMS 191 Method 5514, 1978, or an equivalent thereof.

The topsheet 28 presents a body-facing surface which is compliant, soft-feeling, and non-irritating to the wearer's skin. Further, the topsheet 28 can be less hydrophilic than absorbent body 32, and is sufficiently porous to be liquid permeable, permitting liquid to readily penetrate through its thickness to reach the absorbent body. A suitable topsheet layer 28 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The topsheet layer 28 is typically employed to help isolate the wearer's skin from liquids held in absorbent body 32.

Various woven and nonwoven fabrics can be used for topsheet 28. For example, the topsheet may be composed of a meltblown or spunbonded web of the desired fibers, and may also be a bonded-carded-web. The various fabrics can be composed of natural fibers, synthetic fibers or combinations thereof.

For the purposes of the present description, the term “nonwoven web” means a web of fibrous material which is formed without the aid of a textile weaving or knitting process.

The term “fabrics” is used to refer to all of the woven, knitted and nonwoven fibrous webs.

The topsheet fabrics may be composed of a substantially hydrophobic material, and the hydrophobic material may optionally be treated with a surfactant or otherwise processed to impart a desired level of wettability and hydrophilicity. In a particular embodiment of the invention, topsheet 28 is a nonwoven, spunbond polypropylene fabric composed of about 2.8-3.2 denier fibers formed into a web having a basis weight of about 22 gsm and density of about 0.06 gm/cc. The fabric can be surface treated with an operative amount of surfactant, such as about 0.28% TRITON X-102 surfactant. The surfactant can be applied by any conventional means, such as spraying, printing, brush coating or the like.

The topsheet 28 and backsheet 30 are connected or otherwise associated together in an operable manner. As used herein, the term “associated” encompasses configurations in which topsheet 28 is directly joined to backsheet 30 by affixing topsheet 28 directly to backsheet 30, and configurations wherein topsheet 28 is indirectly joined to backsheet 30 by affixing topsheet 28 to intermediate members which in turn are affixed to backsheet 30. Topsheet 28 and backsheet 30 can, for example, be joined to each other in at least a portion of the diaper periphery by suitable attachment mechanisms (not shown) such as adhesive bonds, sonic bonds, thermal bonds, pinning, stitching or any other attachment technique known in the art, as well as combinations thereof. For example, a uniform continuous layer of adhesive, a patterned layer of adhesive, a sprayed pattern of adhesive or an array of separate lines, swirls or spots of construction adhesive may be used to affix the topsheet 28 to the backsheet 30. It should be readily appreciated that the above-described attachment means may also be employed to suitably interconnect, assemble and/or affix together the various other component parts of the articles which are described herein.

The absorbent body 32 provides an absorbent structure which can include a retention portion, such as the representatively shown absorbent pad composed of selected hydrophilic fibers and high-absorbency particles, for holding and storing absorbed liquids and other waste materials. The absorbent body is positioned and sandwiched between the topsheet 28 and backsheet 30 to form the diaper 10. The absorbent body has a construction which is generally compressible, conformable, non-irritating to the wearer's skin, and capable of absorbing and retaining body exudates. It should be understood that, for purposes of this invention, the absorbent body structure may comprise a single, integral piece of material, or alternatively, may comprise a plurality of individual separate pieces of material which are operably assembled together.

Various types of wettable, hydrophilic fibrous material can be used to form the component parts of absorbent body 32. Examples of suitable fibers include naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers; and synthetic fibers composed of a nonwettable thermoplastic polymer, such as polypropylene fibers, which have been hydrophilized by appropriate means. The fibers may be hydrophilized, for example, by treatment with silica, treatment with a material which has a suitable hydrophilic moiety and is not readily removable from the fiber, or by sheathing the nonwettable, hydrophobic fiber with a hydrophilic polymer during or after the formation of the fiber. For the purposes of the present invention, it is contemplated that selected blends of the various types of fibers mentioned above may also be employed.

As used herein, the term “hydrophilic” describes fibers or the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with such system, fibers having contact angles less than 90° are designated “wettable”, while fibers having contact angles greater than 90° are designated “nonwettable”.

The absorbent body structure 32 can comprise a matrix of hydrophilic fibers, such as a web of cellulosic fluff, mixed with particles of high-absorbency material. In particular arrangements, absorbent body 32 may comprise a mixture of superabsorbent hydrogel-forming particles and synthetic polymer meltblown fibers, or a mixture of superabsorbent particles with a fibrous coform material comprising a blend of natural fibers and/or synthetic polymer fibers. The superabsorbent particles may be substantially homogeneously mixed with the hydrophilic fibers, or may be nonuniformly mixed. For example, the concentrations of superabsorbent particles may be arranged in a non-step-wise gradient through a substantial portion of the thickness (z-direction) of the absorbent structure, with lower concentrations toward the bodyside of the absorbent body and relatively higher concentrations toward the outerside of the absorbent structure. Suitable z-gradient configurations are described in U.S. Pat. No. 4,699,823 issued Oct. 13, 1987 to Kellenberger et al., the entire disclosure of which is incorporated herein by reference in a manner that is consistent (not in conflict) with the present description. Alternatively, the concentrations of superabsorbent particles may be arranged in a non-step-wise gradient, through a substantial portion of the thickness (z-direction) of the absorbent structure, with higher concentrations toward the bodyside of the absorbent body and relatively lower concentrations toward the outerside of the absorbent structure. The superabsorbent particles may also be arranged in a generally discrete layer within the matrix of hydrophilic fibers. In addition, two or more different types of superabsorbent may be selectively positioned at different locations within or along the fiber matrix.

The high-absorbency material may comprise absorbent gelling materials, such as superabsorbents. Absorbent gelling materials can be natural, synthetic and modified natural polymers and materials. In addition, the absorbent gelling materials can be inorganic materials, such as silica gels, or organic compounds such as cross-linked polymers. The term “cross-linked” refers to any means for effectively rendering normally water-soluble materials substantially water insoluble but swellable. Such means can include, for example, physical entanglement, crystalline domains, covalent bonds, ionic complexes and associations, hydrophilic associations, such as hydrogen bonding, and hydrophobic associations or Van der Waals forces.

Examples of synthetic absorbent gelling material polymers include the alkali metal and ammonium salts of poly(acrylic acid) and poly (methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further polymers suitable for use in the absorbent body include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and the like. Mixtures of natural and wholly or partially synthetic absorbent polymers can also be useful in the present invention. Other suitable absorbent gelling materials are disclosed by Assarsson et al. in U.S. Pat. No. 3,901,236 issued Aug. 26, 1975. Processes for preparing synthetic absorbent gelling polymers are disclosed in U.S. Pat. No. 4,076,663 issued Feb. 28, 1978 to Masuda et al. and U.S. Pat. No. 4,286,082 issued Aug. 25, 1981 to Tsubakimoto et al.

Synthetic absorbent gelling materials typically are xerogels which form hydrogels when wetted. The term “hydrogel”, however, has commonly been used to also refer to both the wetted and unwetted forms of the material.

With reference to the representative configurations shown in FIGS. 1 and 2, the article can include a system of flap regions, wings, “ear” regions or ear members. In particular arrangements, each flap, wing or ear region or member may extend laterally at the opposed, lateral ends of the article such as a diaper, incontinence pad or sanitary napkin.

In the various configurations of the invention, the ear, tab, flap or wing regions may be integrally formed with a selected article component. For example, ear, tab, flap or wing regions can be integrally formed from the layer of material which provides backsheet layer and/ or may be integrally formed from the material employed to provide topsheet. In alternative configurations, the ear, tab, flap or wing regions may be provided by one or more separately provided members that are connected and assembled to the backsheet, to the topsheet, in between the backsheet and topsheet, or in various fixedly attached combinations of such assemblies.

In particular configurations of the invention, each of the ear, tab, flap or wing regions may be formed from a separately provided piece of material which is then suitably assembled and attached to a selected portion of the article.

The ear, tab, flap or wing regions may be composed of a substantially non-elastomeric material, such as polymer films, woven fabrics, nonwoven fabrics or the like, as well as combinations thereof. In particular aspects of the invention, ear, tab, flap or wing regions may be composed of a substantially elastomeric material, such as a stretch-bonded-laminate (SBL) material, a neck-bonded-laminate (NBL) material, an elastomeric film, an elastomeric foam material, or the like, which is elastomerically stretchable at least along the lateral direction 24. For example, suitable meltblown elastomeric fibrous webs for forming ear, tab, flap or wing regions are described in U.S. Pat. No. 4,663,220 issued May 5, 1987 to T. Wisneski et al., the entire disclosure of which is hereby incorporated by reference. Examples of composite fabrics comprising at least one layer of nonwoven textile fabric secured to a fibrous elastic layer are described in European Patent Application EP 0 217 032 A2 published on Apr. 8, 1987 which has the listed inventors of J. Taylor et al., the entire disclosure of which is hereby incorporated by reference.

Examples of NBL materials are described in U.S. Pat. No. 5,226,992 issued Jul. 13, 1993 to Mormon, the entire disclosure of which is hereby incorporated by reference in a manner that is consistent herewith.

As previously mentioned, various suitable constructions can be employed to attach the ear, tab, flap or wing regions to the selected portions of the article. Particular examples of suitable constructions for securing a pair of elastically stretchable members to the lateral, side portions of an article to extend laterally outward beyond the laterally opposed side regions of the outer cover and liner components of an article can be found in U.S. Pat. No. 4,938,753 issued Jul. 3, 1990 to P. VanGompel et al., the entire disclosure of which is hereby incorporated by reference in a manner that is consistent herewith.

The illustrated ear, tab, flap or wing regions have a tapered, curved or otherwise contoured shape in which the longitudinal length of the relatively inboard base region is larger or smaller than the longitudinal length of its relatively outboard end region. Alternatively, the ear regions may have a substantially rectangular shape, and optionally may have a substantially trapezoidal shape.

In the various aspects and configurations of the invention, the fastening mechanism between the selected first fastener component and the selected, cooperating fastener component may be adhesive, cohesive, mechanical or combinations thereof. In the context of the present invention, a mechanical fastening system is a system which includes a first fastener component and cooperating fastener component which mechanically inter-engage to provide a desired securement.

Desirably, the first fastener component and cooperating fastener components include complementary elements of a cooperatively interengaging mechanical fastening system. The mechanical fastener components can be provided by mechanical-type fasteners such as hooks, buckles, snaps, buttons and the like, which include cooperating and complementary, mechanically interlocking components.

As shown in the illustrated arrangements, for example, the mechanical fastening system may be a hook-and-loop type of fastening system. Such fastening systems typically include engagement members having the form of a “hook” or hook-like, male component, and include a cooperating “loop” or loop-like, female component which engages and releasably interconnects with the hook component. Desirably, the interconnection is selectively releasable and re-attachable. Conventional systems are, for example, available under the VELCRO trademark. The hook element may be provided by a single-prong hook configuration, a multiple-prong hook configuration or by a generally continuous, expanded-head configuration, such as provided by a mushroom-head type of hook element. The loop element may be provided by a woven fabric, a nonwoven fabric, a knitted fabric, a perforated or apertured layer, and the like, as well as combinations thereof. The many arrangements and variations of such fastener systems have been collectively referred to as hook-and-loop fasteners.

A configuration which employs a selectively releasable, interengaging mechanical fastening system can, for example, locate the first fastener component on at least the appointed mating or securing surface of the tab, flap or wing 36, and can locate the cooperating, second fastener component on the appointed engagement surface of the appointed tab, flap or wing 36. For example, with the representatively shown hook-and-loop fastener, the fastening component which is attached to the appointed mating or securing surface of a fastener tab 36 may include a hook type of mechanical engagement element, and the complementary fastening component, which is operably joined and attached to the appointed surface of a fastener tab 36 can include a loop type of fastening element.

It should also be readily apparent that, in the various configurations of the invention, the relative positions and/or materials of the first fastening component and its cooperating, complementary fastening component can be transposed.

Examples of hook-and-loop fastening systems and components are described in U.S. Pat. No. 5,019,073 issued May 28, 1991 to T. Roessler et al., the entire disclosure of which is hereby incorporated by reference in a manner that is consistent herewith. Other examples of hook-and-loop fastening systems are described in U.S. patent application Ser. No. 366,080 entitled HIGH-PEEL TAB FASTENER, filed Dec. 28, 1994 by G. Zehner et al. (attorney docket No.11,571) which corresponds to U.S. Pat. No. 5,605,735; and U.S. patent application Ser. No. 421,640 entitled MULTI-ATTACHMENT FASTENING SYSTEM, filed Apr. 13, 1995 by P. VanGompel et al.; the entire disclosures of which are hereby incorporated by reference in a manner that is consistent herewith. Examples of fastening tabs constructed with a carrier layer are described in U.S. patent application Ser. No. 08/603,477 of A. Long et al., entitled MECHANICAL FASTENING SYSTEM WITH GRIP TAB and filed Mar. 6, 1996 (attorney docket No.12,563) which corresponds to U.S. Pat. No. 5,624,429 which issued Apr. 29, 1997, the entire disclosure of which is hereby incorporated by reference in a manner which is consistent herewith.

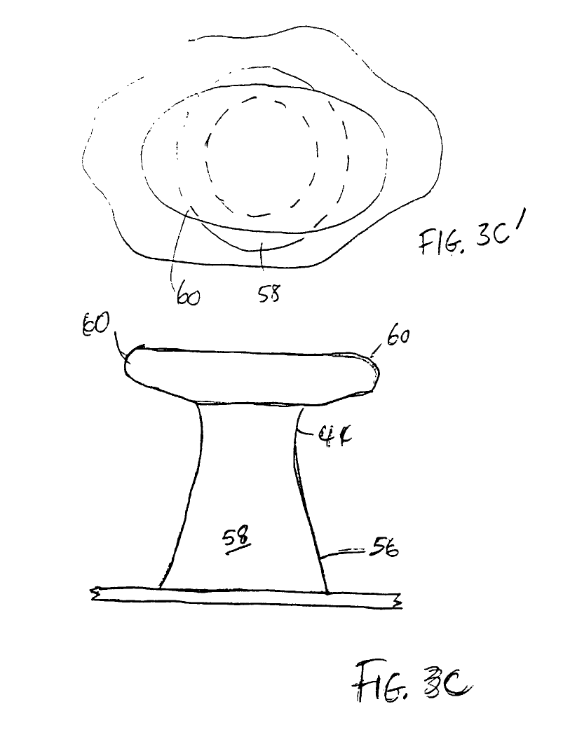

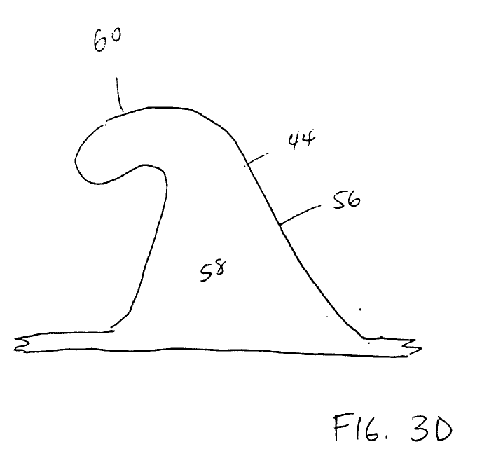

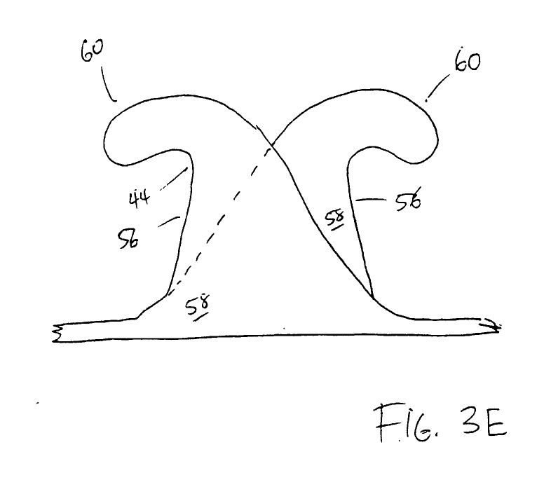

With reference to FIGS. 3A-E, the appointed first fastener component 70 can include a material having engagement members (e.g. the shown hook members) which project away from a base or substrate layer 110. Each engagement member includes a generally, up-standing stem portion 58 and a securement element 60. The stem portion 58 has a fixed end region 43, and a distal end region 44 which, desirably, is contiguously joined with the fixed end region. The fixed end region of the stem portion is operably attached to the substrate layer 110, and the distal end region is operably attached to its corresponding, associated securement element 60. The stem portion 58 is sufficiently rigid to maintain the appointed upright positioning and the appointed directional alignment of the securement element 60 during the ordinary operation of the first fastener component in the fastener system. More particularly, the stem portion is sufficiently resistant to bending and twisting to operably maintain the desired upright positioning and directional alignment of the securement element. The substrate layer 110 has a substrate thickness 112, an engagement member surface 114, and an opposed substrate mounting surface 116. The selected engagement members are attached to the substrate layer 110, and project away from the engagement member surface 114.

As representatively shown in FIGS. 3A-E, particular aspects of the invention may incorporate non-isotropic engagement members where the non-isotropic engagement members are configured to provide a directional or direction-dependent engagement with the cooperating fastener component 72. In particular, the engagement members can exhibit at least one bias direction along which a selected fastening property, such as peel force, shear force or the like, has a relatively different value. For example, the fastening property may have at least one bias direction along which a fastening property, such as peel force, shear force or the like, has a relatively maximal value. Similarly, the engagement members can exhibit at least one bias direction along which the selected fastening property has a relatively minimal value. The direction of maximal value may or may not be substantially opposite to the direction of relatively minimal value.

Thus, the non-isotropic engagement member may provide a greater (or lesser) shear force value or peel force value depending upon the direction along which the shear force or peel force value is determined. The non-isotropic feature may be generated by various suitable mechanisms, such as a difference in shape, size dimension, contour, length of projection, angle of projection, type of material, type of coating or other treatment, surface texture, surface topography, coefficient of friction, cohesion or the like, as well combinations thereof. The non-isotropic engagement member may have a limited degree of symmetry, such as a bilateral symmetry. Suitable non-isotropic engagement members can, for example, be provided by inverted-J shaped or generally T-shaped engagement members. In contrast, substantially isotropic engagement members may be provided by mushroom shaped engagement members where the mushroom head is substantially symmetrically distributed about its upstanding stem portion and where the appointed engagement opening is similarly substantially symmetrically distributed about its upstanding stem portion.

An example of a suitable micro-hook material is distributed under the designations VELCRO HTH 829 and VELCRO HTH 851 and is available from VELCRO U.S.A., Inc., a business having offices in Manchester, N.H. The micro-hook material can have hooks in the shape of angled hook elements, and can be configured with a hook density of about 264 hooks per square centimeter (about 1700 hooks per square inch); a hook height which is within the range of about 0.030-0.063 cm (about 0.012-0.025 inch); and a hook width which is within the range of about 0.007 to 0.022 cm (about 0.003 to 0.009 inch). The hook elements are molded onto a base layer substrate having a thickness of about 0.0076-0.008 cm (about 0.003-0.0035 inch), and the member of hook material has a Gurley stiffness of about 12 mgf (about 12 Gurley units). Other suitable hook materials can include VELCRO HTH 858, VELCRO HTH 851 and VELCRO HTH 863 hook materials.

For the purposes of the present invention, the various stiffness values are determined with respect to a bending moment produced by a force which is directed perpendicular to the plane substantially defined by the length and width of the component being tested. A suitable technique for determining the stiffness values described herein is a Gurley Stiffness test, a description of which is set forth in TAPPI Standard Test T 543 om-94 (Bending Resistance of Paper (Gurley type tester)). A suitable testing apparatus is a Gurley Digital Stiffness Tester; Model 4171-D manufactured by Teledyne Gurley, a business having offices in Troy, N.Y. For purposes of the present description, the stated Gurley stiffness values are intended to correspond to the values that would be generated by a “standard” sized sample. Accordingly, the scale readings from the Gurley stiffness tester are appropriately converted to the stiffness of a standard size sample, and are traditionally reported in terms of milligrams of force (mgf). Currently, a standard “Gurley unit” is equal to a stiffness value of 1 mgf, and may equivalently be employed to report the Gurley stiffness.

In the various aspects and configurations of the invention, the loop material can be provided by a nonwoven, woven or knit fabric. For example, a suitable loop material fabric can be composed of a 2 bar, warp knit fabric of the type available from Guilford Mills, Inc., Greensboro, N.C. under the trade designation #34285, as well as other types of knit fabrics. Suitable loop materials are also available from the 3M Company, which has distributed a nylon woven loop under their SCOTCHMATE brand. The 3M Company has also distributed a linerless loop web with adhesive on the backside of the web, and 3M knitted loop tape.

The loop material may also include a nonwoven fabric having continuous bonded areas defining a plurality of discrete unbonded areas. The fibers or filaments within the discrete unbonded areas of the fabric are dimensionally stabilized by the continuous bonded areas that encircle or surround each unbonded area, such that no support or backing layer of film or adhesive is required. The unbonded areas are specifically designed to afford spaces between fibers or filaments within the unbonded area that remain sufficiently open or large to receive and engage hook elements of the complementary hook material. In particular, a pattern-unbonded nonwoven fabric or web may include a spunbond nonwoven web formed of single component or multi-component melt-spun filaments. At least one surface of the nonwoven fabric can include a plurality of discrete, unbonded areas surrounded or encircled by continuous bonded areas. The continuous bonded areas dimensionally stabilize the fibers or filaments forming the nonwoven web by bonding or fusing together the portions of the fibers or filaments that extend outside of the unbonded areas into the bonded areas, while leaving the fibers or filaments within the unbonded areas substantially free of bonding or fusing. The degree of bonding or fusing within the bonding areas desirably is sufficient to render the nonwoven web non-fibrous within the bonded areas, leaving the fibers or filaments within the unbonded areas to act as “loops” for receiving and engaging hook elements. Examples of suitable point-unbonded fabrics are described in U.S. patent application Ser. No. 754,419 entitled PATTERN-UNBONDED NONWOVEN WEB AND PROCESS FOR MAKING THE SAME, by T. J. Stokes et al., and filed Dec. 17, 1996 (attorney docket No. 12,232); the entire disclosure of which is incorporated herein by reference in a manner that is consistent herewith.

As used herein, the term “spunbond web” refers to a web formed by extruding a molten thermoplastic material as filaments from a plurality of fine, usually circular, capillaries with the diameter of the extruded filaments then being rapidly reduced, for example, by fluid-drawing or other well known spunbonding mechanisms. The production of spunbond nonwoven webs is illustrated in patents such as Appel, et al., U.S. Pat. No. 4,340,563.

In the various configurations of the invention, the loop material need not be limited to a discrete or isolated patch on the outward surface of the article. Instead, the loop material can be provided by a substantially continuous, outer fibrous layer which is assembled, integrated or otherwise joined to extend over a predetermined surface area of the desired article. For example, the outer fibrous layer may be arranged to extend over substantially the total exposed surface area of a cloth-like outer cover employed with the article.

In the various configurations of the invention, the engagement force between the selected first fastener component and its appointed and cooperating second fastener component should be large enough and durable enough to provide an adequate securement of the article on the wearer during use. In particular arrangements, especially where there are sufficiently high levels of engagement shear force provided by the fastening system, the fastening engagement may provide a peel force value of not less than a minimum of about 40 grams-force (gmf) per inch of the “width” of engagement between the first and second fastener components. In further arrangements, the fastening engagement may provide a peel force value of not less than about 100 gmf/inch to provide improved advantages. In desired configurations, the fastening engagement may provide a peel force value of not less than about 200 gmf per inch of the “width” of engagement between the first and second fastener components . Alternatively, the peel force is not less than about 300 gmf/inch, and optionally is not less than about 400 gmf/inch to further provide improved benefits. In other aspects, the peel force is not more than about 1,200 gmf/inch. Alternatively, the peel force is not more than about 800 gmf/inch, and optionally is not more than about 600 gmf/inch to provide improved performance.

The engagement force between the selected first fastener component and its appointed and cooperating second fastener component may additionally provide a shear force value of not less than about 400 gmf per square inch of the area of engagement between the first and second fastener components. Alternatively, the shear force is not less than about 1,000 gmf/in², and optionally, is not less than about 1,700 gmf/in². In further aspects, the shear force can be up to about 4,400 gmf/in², or more. Alternatively, the shear force is not more than about 3,900 gmf/in², and optionally is not more than about 3,500 gmf/in² to provide improved performance.

The peel force value can be determined in accordance with standard procedure ASTM D-5170, approved Sep. 15, 1991 and published November 1991; with the following particulars. The test specimen is the fastener tab from the article being assessed. The test specimen length is the dimension aligned along the direction in which a peel-away force is typically applied to disengage and remove the fastener during the ordinary use of the article with which the fastener is employed. The specimen “width” lies within the general plane of the fastener and is perpendicular to the specimen length. The roller device weighs 4.5 pounds and includes a rubber coating around the roller circumference. A suitable roller is part number HR-100 available from Chemsultants International, a business having a location in Mentor, Ohio. During the engagement of the fastener components, the roller is rolled over the test specimen through one cycle in the direction of the cross-wise “width” of the sample. In addition, the initial peel by hand to “raise the loops” is omitted. During testing, the fastener material held by the stationary clamp can be larger in area, as compared to the fastener material held in the moving clamp. The initial separation distance between the clamps of the tensile tester is 4 inch, and the extension speed of the tensile testing machine is 20 inch/min. The reported value of a peel test result is a “three-peak average” value employing MTS TESTWORKS software with a peak criteria of 2%. Additionally, the peel force value is normalized to be stated in terms of force per unit length of the “width” dimension of the fastener component on the test specimen, such as grams per inch. The MTS TESTWORKS software is available from MTS Systems Corporation, a business having offices in Eden Prairie, Minn.

The shear force value can be determined in accordance with the standard procedure ASTM D-5169, approved Sep. 15, 1991 and published November 1991 with the following particulars. The test specimen is composed of the fastener tab from the article being assessed. The test specimen length and width typically correspond to the length and width employed to conduct the testing for peel force value. Ordinarily, the test specimen length is the dimension aligned along the direction in which a shear force is typically applied to the fastener during the ordinary use of the article with which the fastener is employed. The specimen “width” lies within the general plane of the fastener and is perpendicular to the specimen length. The roller device weighs 4.5 pounds and includes a rubber coating around the roller. A suitable roller is part number HR-100 available from Chemsultants International, a business having a location in Mentor, Ohio. During the engagement of the fastener components, the roller is rolled over the test specimen through five cycles in the direction of the cross-wise “width” of the sample. In addition, the initial peel by hand to “raise the loops” is omitted. During testing, the fastener material (e.g. the loop material) held by the stationary clamp can be larger in area, as compared to the fastener material (e.g. hook material) held in the moving clamp. The initial separation distance between the clamps of the tensile tester is 4 inch, and the extension speed of the tensile testing machine is 10 inch/min. The shear force value is normalized to be stated in terms of force per unit area of the test specimen, such as grams per inch².

The particulars of the standard test procedures are intended to generate fastening conditions that can be more representative of consumer use conditions. When preparing the test specimen materials (e.g. hook and loop materials) to determine the cooperating peel and/or shear force values for the representatively shown configurations of the invention, it should be noted that, the width dimension of the selected specimen material will correspond to the dimension of the fastener material which, in the actual article, is found to be aligned along the longitudinal direction 26 of the article. Similarly, the length dimension of the selected specimen material will correspond to the dimension of the fastener material which, in the actual article, is found to be aligned along the lateral direction 24 of the article.

Desirably, the securing engagement between the first fastener component and the cooperating fastener components should be sufficient to prevent a disengagement of the components when subject to a tensile force of at least about 1,000 grams when the tensile force is applied outwardly along the lateral direction, aligned generally parallel with the plane of the backsheet layer of the article.

Each of the fastener components and fastening elements in the various constructions of the invention may be operably attached to its supporting substrate by employing any one or more of the attachment mechanisms employed to construct and hold together the various other components of the article of the invention. The fastening elements in the various fastening regions, may be integrally formed, such as by molding, co-extrusion or the like, along with their associated substrate layer. The substrate layer and its associated mechanical fastening elements may be formed from substantially the same polymer material, and there need not be a discrete step of attaching the fastening elements to an initially separate substrate layer. For example, the individual hook elements may be integrally formed simultaneously with a hook base-layer by coextruding the base layer and hook elements from substantially the same polymer material.

It should be readily appreciated that the strength of the attachment or other interconnection between the substrate layer and the attached fastening component should be greater than the peak force required to remove the fastener tab 36 from its releasable securement to the appointed landing member of the article.

### EXAMPLES

The following examples are presented to provide a more detailed understanding of the invention, and are not intended to specifically limit the scope of the invention.

Peel testing was conducted generally in accordance with ASTMD-5170-91 and shear testing was conducted generally in accordance with ASTM D-5169-91. Each test utilized 10 mm squares of HTH-851 hook material available from VELCRO hand-mounted on 2.0osy PRESTO PUB loop material (See U.S. application Ser. No. 754,419). The hook material was oriented in either MD or CD for the hook direction. The pads were worn by a mechanical walking model for 30 minutes at 72 strides/minute.

### Equipment

Tensile Tester—Constant Rate of Extension tensile tester with MTS TestWorks software.

Walking Model (Size 5 torso)

Fruit of the Loom Women's Underwear (Size 5)

### Sample Preparation

For Standard Peel and Shear testing:

Wings are removed from the product cutting along the line of adhesive juncture.

Wings are then engaged used a mechanical roller to consistently engage wings Mechanical roller available from Cheminstruments with a 2 Kg weight.

Sample is then ready for Peel or Shear testing.

For post wear testing of Peel and Shear:

Underwear are placed on Walking Model.

Pad is placed in underwear. Edge of front lobe always 1 cm in front of the crotch seam.

Wings are then engaged and the underwear pulled onto the model.

Model is set to walk at 72 strides per minute for 30 minutes.

Underwear are cut off the model and removed carefully to not impact hook engagement.

Wings are the removed from product and panty by cutting along the adhesive juncture.

Sample is the ready for post wear Peel and Shear testing.

### Peel Test

Finger tab (area at end of wing beyond hook is placed in the upper jaw, cut edge of other wing is place in the lower jaw. Careful not to leave too much slack or pull hook from loop engagement

Test is run at the following parameters:

Crosshead speed—20 in/min

Gauge Length—3 inches

Load units—Grams

Start Measure—0.4 in

End Measure—0.9 in

Break Sensitive—110%

Slack Compensation—50 grams

Response is the averaged gram of force over the length of the peel test.

### Shear Test

Cut edge of one wing is placed in the upper jaw and cut edge of opposing wing is placed in the lower jaw. Careful not to leave too much slack or pull hook from loop engagement.

Test is run at the following parameters:

Crosshead speed—20 in/min

Gauge Length—3 inches

Load units—Grams

Start Measure—0.4 in

End Measure—0.9 in

Break Sensitive—110%

Slack Compensation—50 grams

Response is the averaged gram of force over the length of the peel test.

Results of testing is reported in Table 1.

TABLE 1

| | MD- | CD- | MD- | CD- | MD- | CD- | MD- | CD- |
|---|---|---|---|---|---|---|---|---|
| | Peel | Peel | Shear | Shear | Peel | Peel | Shear | Shear |
| | Std | Std | Std | Std | Post | Post | Post | Post |
| | Test | Test | Test | Test | Wear | Wear | Wear | Wear |
| | 1.71 | 7.22 | 512.9 | 603.8 | 74.7 | 32.96 | 437 | 674 |
| | 4.27 | 14.02 | 237.9 | 491 | 35.92 | 12.2 | 635.8 | 649.6 |
| | 0.1 | 0.94 | 201 | 785.2 | 42.57 | 15.8 | 469.7 | 454.4 |
| | 9.65 | 8.38 | 318 | 618.8 | 36.86 | 23.16 | 1125.7 | 551.4 |
| | 6.9 | 1.88 | 202 | 591.9 | 206.0 | 26.26 | 1208.8 | 485.6 |
| | 6.53 | 10.37 | 267.9 | 435 | 3 | 23.49 | 510.8 | 519.7 |
| | 1.61 | 19.63 | 329.9 | 473 | 34.78 | 13.74 | 685.3 | 455.8 |
| | 6.39 | 13.8 | 272.7 | 912.7 | 72.98 | 33.29 | 566.7 | 554.7 |
| | 19.72 | 29.26 | 550.8 | 427.2 | 57.75 | 25.35 | 780.9 | 581 |
| | 1.46 | 9.38 | 341.4 | 484.4 | 78.67 | 16.29 | 934.5 | 383.1 |
| | 7.42 | 4.24 | 403.7 | 610.4 | 67.47 | 43.95 | 382.3 | 466.6 |
| | 3.75 | 17.86 | 237.4 | 400.4 | 54.37 | 29.39 | 523.9 | 434.5 |
| | 9.69 | 20.62 | 246.4 | 567.5 | 54.5 | 32.2 | 740.1 | 299.5 |
| | 8.31 | 13.53 | 408.3 | 525.6 | 66.31 | | 619.1 | 438.7 |
| | 8.07 | 5.74 | 424.2 | 407.3 | 23.65 | | 771.5 | 425.9 |
| | | | | | 47.73 | | | |
| Average | 6.4 | 11.8 | 330.3 | 555.6 | 63.6 | 25.2 | 692.8 | 491.6 |
| Std | 4.8 | 7.8 | 109.3 | 142.7 | 42.7 | 9.2 | 242.9 | 98.9 |
| Dev. | | | | | | | | |

The data are summarized in Table 2. For both shear and peel values, the orientation having the lowest weakest initial MD and CD peel and shear strength values (i.e., the ‘illogical’ orientation) engaged strongly during use. In contrast, the orientation having the greatest or strongest initial MD and CD peel and shear strength values (i.e., the “logical” orientation) either strengthens somewhat during wear or falls in strength. Also, for both the shear and peel value, the absolute after wear values are higher for the ‘illogical’ orientation than for the ‘logical’.

TABLE 2

| | before | | | | ratio, before |
|---|---|---|---|---|---|
| | wear | SD | after wear | SD | to after |
| illogical (MD) peel | 6.4 | 4.8 | 64 | 43 | 10.0 |
| logical (CD) peel | 11.8 | 7.8 | 25 | 9 | 2.1 |
| illogical (MD) shear | 330 | 109 | 693 | 242 | 2.1 |
| logical (CD) shear | 556 | 143 | 491 | 99 | 0.9 |

These examples illustrate an embodiment of the mechanical fastening system of the present invention. More particularly, these examples show an embodiment in which an absorbent article such as, for example, a sanitary napkin has a fastening system that includes a pair of wings including selectively releasable, interengaging fasteners such that the wings are adapted to hold the sanitary napkin to an undergarment. The wings are adapted to increase engagement during use.

Having described the invention in rather full detail, it will be readily apparent that various changes and modifications can be made without departing from the spirit of the invention

We claim:

1. A selectively releasable, interengaging fastening system comprising:
a first fastener component comprising at least a first portion of an article, the first fastener component including an engagement section having a plurality of substantially non-isotropic engagement members such that the engagement section has an axis of substantially maximal engagement; and
a cooperating fastener component comprising at least a second portion of an article such that the first and second portions of the article are capable of being joined by movement of the respective components together generally along an attachment direction into an overlapping and interengaging configuration;
wherein the first fastener component is oriented so its axis of substantially maximal engagement is generally orthogonal to the attachment direction.

2. The fastening system of claim 1, wherein each substantially non-isotropic engagement member has a stem portion with a distal end region and a securement element disposed at the distal end region of its corresponding stem portion.

3. The fastening system of claim 1, wherein the engagement section has an axis of maximal engagement and a generally perpendicular axis of minimal engagement.

4. The fastening system of claim 1, wherein the cooperating fastener component is substantially non-isotropic and includes an axis of substantially maximal engagement.

5. The fastening system of claim 4, wherein the axis of substantially maximal engagement of the substantially non-isotropic engagement section and the axis of substantially maximal engagement of the substantially non-isotropic cooperating fastener component are both substantially parallel and each is oriented generally orthogonal to the attachment direction.

6. The fastening system of claim 1, wherein at least one of the first portion and the second portion of the article comprises a segment having a fixed end attached to an article body and a free end.

7. The fastening system of claim 1, wherein the cooperating fastener component is a nonwoven loop material.

8. The fastening system of claim 7, wherein the nonwoven loop material is a pattern unbonded material.

9. The fastening system of claim 1, wherein the fastening system can be used to secure the article in a configuration convenient for disposal.

10. An article, having a lengthwise longitudinal direction, a lateral cross-direction, and a longitudinally extending medial line, said article comprising:
a first article portion; a second article portion; and at least one selectively releasable, interengaging fastener system for securing the first article portion to the second article portion;
the selectively releasable, interengaging fastener system including:
at least one first fastener component comprising at least one section of the first article portion, the first fastener component including an engagement section having a plurality of substantially non-isotropic engagement members such that the engagement section has an axis of substantially maximal engagement;
at least one cooperating fastener component comprising at least one section of the second article portion such that the first and second article portions are capable of being joined by movement of the respective components together generally along an attachment direction into an overlapping and interengaging configuration,
wherein the first fastener component is oriented so its axis of substantially maximal engagement is generally orthogonal to the attachment direction.

11. The article of claim 10, wherein each substantially non-isotropic engagement member has a stem portion with a distal end region and a securement element disposed at the distal end region of its corresponding stem portion.

12. The article of claim 10, wherein the engagement section has an axis of maximal engagement and a generally perpendicular axis of minimal engagement.

13. The article of claim 10, wherein the cooperating fastener component is substantially non-isotropic and includes an axis of substantially maximal engagement.

14. The article of claim 13, wherein the axis of substantially maximal engagement of the substantially non-isotropic engagement section and the axis of substantially maximal engagement of the substantially non-isotropic cooperating fastener component are both substantially parallel and each is oriented generally orthogonal to the attachment direction.

15. The article of claim 10, wherein at least one of the first article portion and the second article portion comprises a segment having a free end and a fixed end attached to an article body.

16. The article of claim 10, wherein the cooperating fastener component is a nonwoven loop material.

17. The article of claim 16, wherein the nonwoven loop material is a pattern unbonded material.

18. The article of claim 10, wherein the first article portion provides a first waistband portion; said second article portion provides a second waistband portion; the article has an intermediate portion which interconnects said first and second waistband portions; and wherein the article further comprises:
a backsheet layer;
a substantially liquid-permeable topsheet layer; and
an absorbent body sandwiched between said backsheet layer and topsheet layer.

19. The article of claim 10, wherein the first article portion and the second article portion each provide respective first and second flap portions, each flap portion having a free end and a fixed end attached to an intermediate portion which interconnects the first and second flap portions; and wherein the article further comprises:
a backsheet layer;
a substantially liquid-permeable topsheet layer; and
an absorbent body sandwiched between said backsheet layer and
topsheet layer.

20. The article of claim 10, wherein the fastening system can be used to secure the article in a configuration convenient for disposal.

21. A selectively releasable, interengaging fastening system for a disposable sanitary napkin, the fastening system comprising:
a first fastener component comprising at least a first portion of the sanitary napkin, the first fastener component including an engagement section having a plurality of substantially non-isotropic engagement members such that the engagement section has an axis of substantially maximal engagement; and
a cooperating fastener component comprising at least a second portion of the sanitary napkin such that the first and second portions of the sanitary napkin are capable of being joined by movement of the respective components together generally along an attachment direction into an overlapping and interengaging configuration;
wherein the first fastener component is oriented so its axis of substantially maximal engagement is generally orthogonal to the attachment direction.

22. The fastening system of claim 21, wherein each substantially non-isotropic engagement member has a stem portion with a distal end region and a securement element disposed at the distal end region of its corresponding stem portion.

23. The fastening system of claim 21, wherein the engagement section has an axis of maximal engagement and a generally perpendicular axis of minimal engagement.

24. The fastening system of claim 21, wherein the cooperating fastener component is substantially non-isotropic and includes an axis of substantially maximal engagement.

25. The fastening system of claim 21, wherein the first portion and the second portion of the sanitary napkin each provide respective first and second flap portions, each flap portion having a free end and a fixed end attached to an intermediate portion which interconnects the first and second flap portions; and wherein the sanitary napkin further comprises:
a backsheet layer;
a substantially liquid-permeable topsheet layer; and
an absorbent body sandwiched between said backsheet layer and topsheet layer.

26. The fastening system of claim 25, wherein at least one of the first and second flap portions comprises the cooperating fastener component

27. The fastening system of claim 26, wherein the cooperating fastener component is a nonwoven loop material.

28. The fastening system of claim 27, wherein the nonwoven loop material is a pattern unbonded material.

29. The fastening system of claim 21, wherein the fastening system can be used to secure the sanitary napkin in a configuration convenient for disposal.

30. A sanitary napkin having a pair of end edges, a first longitudinal edge and a second longitudinal edge located between the end edges, the sanitary napkin a selectively releasable, interengaging fastening system comprising:
a first fastener component comprising at least first a portion of the sanitary napkin adjacent the first longitudinal edge, the first fastener component including an engagement section having a plurality of substantially non-isotropic engagement members such that the engagement section has an axis of substantially maximal engagement; and
a cooperating fastener component comprising at least a second portion of the sanitary napkin adjacent the second longitudinal edge such that the first and second portions of the sanitary napkin are capable of being joined by movement of the respective components together generally along an attachment direction into an overlapping and interengaging configuration;
wherein the first fastener component is oriented so its axis of substantially maximal engagement is generally orthogonal to the attachment direction.

31. The sanitary napkin of claim 30, wherein the first portion of the sanitary napkin is a first wing that extends from the first longitudinal edge of the sanitary napkin, the first wing having a fixed end and a free end.

32. The sanitary napkin of claim 31, wherein the first wing comprises the first fastener component and the second fastener component.

33. The sanitary napkin of claim 32, wherein the second fastener component is a nonwoven loop material.

34. The sanitary napkin of claim 33, wherein the nonwoven loop material is a pattern unbonded material.

35. The sanitary napkin of claim 30, wherein the second portion of the sanitary napkin is a second wing that extends from the second longitudinal edge of the sanitary napkin, the second wing having a fixed end and a free end.

36. The sanitary napkin of claim 35, wherein the second wing comprises the first fastener component and the second fastener component.

37. The sanitary napkin of claim 36, wherein the second fastener component is a nonwoven loop material.

38. The sanitary napkin of claim 37, wherein the nonwoven loop material is a pattern unbonded material.

39. A sanitary napkin having a pair of end edges, a first longitudinal edge and a second longitudinal edge located between the end edges, the sanitary napkin including a selectively releasable, interengaging fastening system comprising:
a first wing extending from the first longitudinal edge of the sanitary napkin and a second wing extending from the second longitudinal edge of the sanitary napkin, each wing having a fixed end and a free end;
a first fastener component comprising at least a portion of at least the first wing, the first fastener component including an engagement section having a plurality of substantially non-isotropic engagement members such that the engagement section has an axis of substantially maximal engagement; and
a cooperating fastener component comprising at least a portion of at least the second wing such that the first and second wings of the sanitary napkin are capable of being joined by movement of the respective components together generally along an attachment direction into an overlapping and interengaging configuration;
wherein the first fastener component is oriented so its axis of substantially maximal engagement is generally orthogonal to the attachment direction.

40. The sanitary napkin of claim 39, wherein the first wing and the second wing include the first fastener component and the second fastener component.

41. The sanitary napkin of claim 39, wherein the second fastener component is a nonwoven loop material.

42. The sanitary napkin of claim 41, wherein the nonwoven loop material is a pattern unbonded material.

43. A sanitary napkin having a pair of end edges, a first longitudinal edge and a second longitudinal edge located between the end edges, the sanitary napkin including a selectively releasable, interengaging fastening system comprising:
a first wing extending from the first longitudinal edge of the sanitary napkin and a second wing extending from the second longitudinal edge of the sanitary napkin, each wing having a fixed end and a free end;
a first fastener component comprising at least a portion of the first wing and the second wing, the first fastener component including an engagement section having a plurality of substantially non-isotropic engagement members such that the engagement section has an axis of substantially maximal engagement; and
a cooperating fastener component comprising at least a portion of the first wing and the second wing such that the first and second wings of the sanitary napkin are capable of being joined by movement of the respective components together generally along an attachment direction into an overlapping and interengaging configuration;
wherein the first fastener component is oriented so its axis of substantially maximal engagement is generally orthogonal to the attachment direction.

44. The sanitary napkin of claim 43, wherein each substantially non-isotropic engagement member has a stem portion with a distal end region and a securement element disposed at the distal end region of its corresponding stem portion.

45. The sanitary napkin of claim 43, wherein the second fastener component is a nonwoven loop material.

46. The sanitary napkin of claim 45, wherein the nonwoven loop material is a pattern unbonded material.

47. The sanitary napkin of claim 43, wherein at least one wing includes perforation lines adjacent its fixed end to provide quick removal of the napkin by tearing the wing along the perforation lines.

48. A selectively releasable, interengaging fastening system for a disposable personal care product, the fastening system comprising:
a first fastener component comprising an engagement section having a plurality of engagement members; and
a cooperating fastener component;
wherein the fastening system is adapted to become more interengaged as the disposable personal care product is worn.
